(19) United States
(12) Patent Application Publication
(10) Pub. No.: US 2024/0055106 A1
(43) Pub. Date: Feb. 15, 2024

(54) DATA PROCESSING APPARATUS AND METHOD

(71) Applicant: CANON MEDICAL SYSTEMS CORPORATION, Otawara-shi (JP)

(72) Inventors: Hannah WATSON, Edinburgh (GB); Alison O'NEIL, Edinburgh (GB); Hamish MACKINNON, Edinburgh (GB)

(73) Assignee: CANON MEDICAL SYSTEMS CORPORATION, Otawara-shi (JP)

(21) Appl. No.: 17/819,116

(22) Filed: Aug. 11, 2022

(51) Int. Cl. G16H 40/20; G16H 10/60

(52) U.S. Cl. CPC G16H 40/20; G16H 10/60

(57) ABSTRACT

A medical information processing system for processing medical data relating to a target patient comprises a memory storing a knowledge base, wherein the knowledge base is based on medical knowledge; and processing circuitry configured to receive trigger information; and determine a necessity of alerting relating to the trigger information based on the trigger information and the knowledge base.

### FIELD

Embodiments described herein relate generally to a data processing apparatus and method, for example an apparatus and method for processing clinical data relating to a patient or other subject.

### BACKGROUND

The amount of data captured for a given patient by hospital systems is ever increasing. Medical records may contain large quantities of data even for a single patient.

Electronic Health Records (EHRs) are becoming commonplace. EHRs may comprise a large variety of types of EHR documents from hundreds of EHR data sources, for example discharge letters, GP (general practitioner) referrals, clinical letters and radiology reports.

An Electronic Health Record relating to a individual patient may comprise a mixture of data types and may include both structured and unstructured (free text) data, plus imaging data. The unstructured data may comprise, for example, clinical notes or letters that comprise free text provided by clinicians. The structured data may comprise vital sign measurement results which may include, for example, measurements of temperature, blood pressure, pulse rate and/or respiration rate. The structured data may comprise laboratory results which may include, for example, the results of blood or urine tests. The structured data may comprise medication data.

It is known for a clinical user, for example a physician, to perform various tasks with relation to stored medical data. For example, the clinical user may enter a search term into a system that highlights all instances of the search term in an unstructured text document. Currently, there may typically be limited search capabilities to navigate an EHR.

Clinical users may typically be overloaded with information and may find it difficult to find key information for a current clinical decision. The use of EHRs may in some circumstances increase a clinician's workload. In general, doctors have limited time to spend on each patient and in some circumstances may miss prescribing relevant investigations or interventions.

In some cases, doctors may miss clinically actionable information. When reviewing the large amount of information that is present in an EHR, there may be a risk of missing key clinical information that might change the doctors intervention or discussion or consultation with the patient. For example, information about patient's risk factors or social history may be missed.

Furthermore, there may be a need to optimize or maximize an opportunity for patient counselling or intervention to occur when the patient is in discussion with the doctor. In some circumstances, patient morbidity and mortality outcomes may be improved by opportunistically prompting the doctor about relevant holistic healthcare actions, for example offering advice or a service for smoking cessation.

### BRIEF DESCRIPTION OF THE DRAWINGS

Embodiments are now described, by way of non-limiting example, and are illustrated in the following figures, in which:

FIG. 1 is a schematic diagram of an apparatus according to an embodiment;

FIG. 2 is a flow chart illustrating in overview a method in accordance with an embodiment;

FIG. 3 is a schematic illustration showing a simplified example of a set of nodes of a knowledge graph; and

FIG. 4 is a flow chart illustrating in overview a method of rule creation in accordance with an embodiment.

### DETAILED DESCRIPTION

Certain embodiments provide a medical information processing system for processing medical data relating to a target patient, the medical information processing system comprising: a memory storing a knowledge base, wherein the knowledge base is based on medical knowledge; and processing circuitry configured to: receive trigger information; and determine a necessity of alerting relating to the trigger information based on the trigger information and the knowledge base.

Certain embodiments provide a method for processing medical data relating to a target patient, the method comprising: receiving trigger information; and determining a necessity of alerting relating to the trigger information based on the trigger information and a knowledge base, wherein the knowledge base is based on medical knowledge.

Certain embodiments provide a medical information processing system for determining rules associating alerting with trigger information, the medical information processing system comprising: a memory storing a knowledge base, wherein the knowledge base is based on medical knowledge; and processing circuitry configured to: receive or determine one or more concepts; determine a subregion of the knowledge base, wherein the determining is based on the one or more concepts; and associate one or more triggers within the subregion with an alert.

Certain embodiments provide a medical information processing method for determining rules associating alerting with trigger information, the method comprising: receiving or determining one or more concepts; determining a subregion of a knowledge base, wherein the determining is based on the one or more concepts and wherein the knowledge base is based on medical knowledge; and associating one or more triggers within the subregion with an alert.

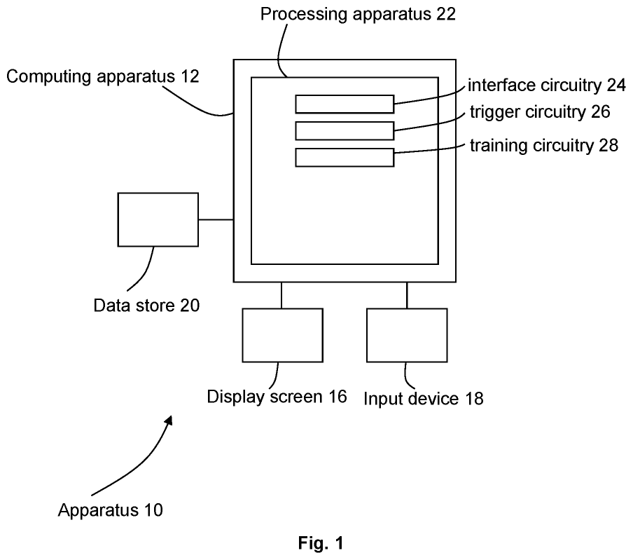

An apparatus 10 according to an embodiment is illustrated schematically in FIG. 1. The apparatus 10 may also be referred to as a medical information processing system.

In the present embodiment, the apparatus 10 is configured to process medical data, for example Electronic Medical Records. The medical data may comprise both structured data and unstructured data. For example, the structured data may comprise vital signs data and/or laboratory data. The unstructured data may comprise free text data such as clinical notes or letters.

In other embodiments, the apparatus 10 may be configured to process any appropriate data, which may comprise non-medical data.

The apparatus 10 comprises a computing apparatus 12, which in this case is a personal computer (PC) or workstation. The computing apparatus 12 is connected to a display screen 16 or other display device, and an input device or devices 18, such as a computer keyboard and mouse.

The computing apparatus 12 receives medical data from a data store 20, which may also be referred to as a memory. In alternative embodiments, computing apparatus 12 receives medical data from one or more further data stores (not shown) instead of or in addition to data store 20. For example, the computing apparatus 12 may receive medical data from one or more remote data stores (not shown) which may form part of an Electronic Medical Records system or Picture Archiving and Communication System (PACS), or which may comprise cloud-based storage.

The data store 20 further comprises a knowledge base which is based on medical knowledge. For example, the knowledge base may be a knowledge graph which comprises knowledge about a large number of medical concepts and their relationships. In other embodiments, the knowledge base may be stored in any suitable memory, for example in another apparatus or in a cloud-based memory.

Computing apparatus 12 provides a processing resource for automatically or semi-automatically processing medical text data. Computing apparatus 12 comprises a processing apparatus 22. The processing apparatus 22 comprises interface circuitry 24 configured to interact with an electronic health record (EHR) and display information to a user, trigger circuitry 26 configured to determine and map triggers, and training circuitry 28 configured to teach or learn mappings between triggers and actions.

In the present embodiment, the circuitries 24, 26, 28 are each implemented in computing apparatus 12 by means of a computer program having computer-readable instructions that are executable to perform the method of the embodiment. However, in other embodiments, the various circuitries may be implemented as one or more ASICs (application specific integrated circuits) or FPGAs (field programmable gate arrays). Functionality of one or more of the circuitries may be implemented in one or more cloud-based computing resources.

The computing apparatus 12 also includes a hard drive and other components of a PC including RAM, ROM, a data bus, an operating system including various device drivers, and hardware devices including a graphics card. Such components are not shown in FIG. 1 for clarity.

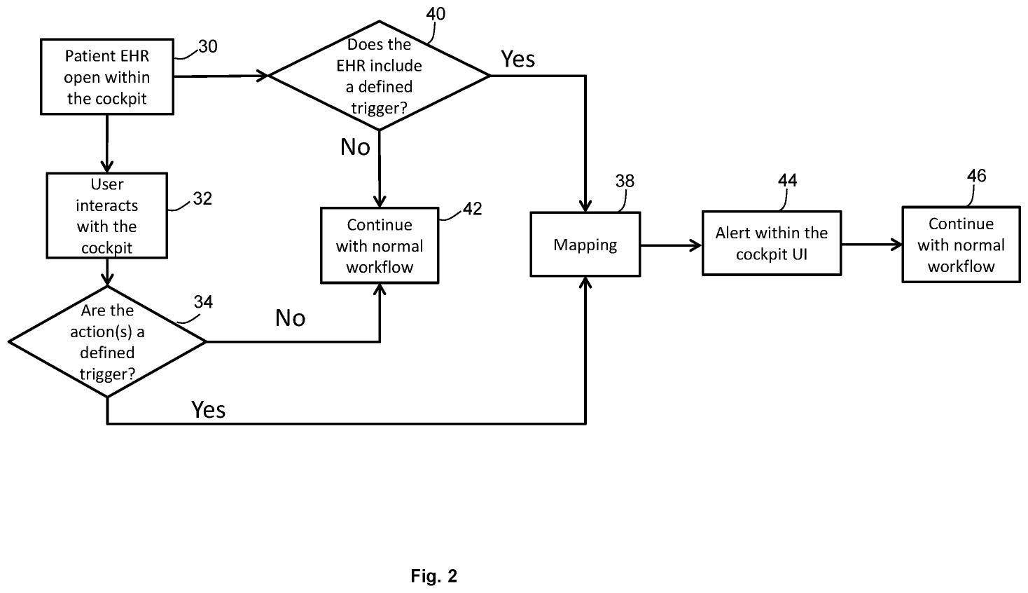

FIG. 2 is a flow chart illustrating in overview a method of an embodiment. The apparatus of FIG. 1 is configured to perform the method of FIG. 2.

At stage 30, the interface circuitry 24 retrieves a set of medical data relating to a patient from data store 20. In the embodiment of FIG. 2, the set of medical data is an electronic health record (EHR). The patient is assigned to a health professional.

The interface circuitry 24 opens the EHR within an EHR viewing application, which may be referred to as a clinical cockpit or as part of a clinical decision support system. The clinical cockpit provides an interface allowing a user to access information including text documents within the EHR. The user may be the health professional to whom the patient is assigned. In other embodiments, the user may be any authorized clinical user.

The interface may allow the user to easily access multiple types of data relating to a single patient, for example by allowing a user to see an overview of the patient and to access detailed information such as symptoms, diagnoses, vital signs and prescriptions.

In other embodiments, the EHR may be opened with any medical EHR software, which may not comprise a clinical cockpit, and the user interacts with the medical EHR software.

The EHR comprises both structured data and unstructured data. The structured data may comprise, for example, at least one of vital signs data, laboratory data, imaging data, medication data, observation data. The unstructured data comprises text data, for example clinical notes, GP records, discharge letters, outpatient letters, or inpatient records. The EHR may comprise imaging data and radiology reports.

In other embodiments, the set of medical data may comprise any suitable medical data, for example one or more of a clinical note, a nursing note, a set of imaging data, a set of imaging measurements, a set of lab result data, a set of patient observation data, a set of vital sign data, a prescription, a medication record, data obtained from the patient, data obtained from a medical device, a summary report, a medical history report, a case conference report, a billing report, a radiology report, a set of patient events, medication data, administration or records data or any other suitable set of recorded information relating to the patient.

In the present embodiment the EHR is obtained from data store 20. In other embodiments, medical data may be obtained from any suitable data store or data stores, for example from multiple servers on a network. The medical data may be obtained from cloud storage. The medical data may be gathered from a healthcare informatics system or from a variety of healthcare informatics systems. The medical data may be formatted in any suitable electronic format, for example any known format for electronic medical record data. DICOM structured reports are one such possible format. In some embodiments, data pertaining to different types of medical data may have different data formats.

At stage 32, an authorized clinical user, for example a physician, interacts with the cockpit by performing one or more user actions. In other embodiments, the user may be any suitable user, for example any suitable medical professional or researcher. The interface circuitry 24 receives user input data that is indicative of the one or more user actions.

The one or more user actions may comprise, for example, inputting a search term to perform a keyword search; opening a document or combination of documents; prescribing a medication, for example warfarin; opening or filling out a checklist, for example a thrombolysis checklist; or reviewing an image. The document or combination or documents may be multimodal. For example, a combination of an ECHO (echocardiogram) and a cardiac clinical letter may indicate that the user is interested in cardiology workflows.

At stage 34, the trigger circuitry 26 processes the user input data and determines whether the one or more actions comprise at least one defined trigger. Defined triggers obtained from actions of a clinical user may also be referred to as defined user triggers. In the present embodiment, a set of defined user triggers is stored in the apparatus, for example, in the data store 20 or in any suitable data store. The trigger circuitry 26 determines whether any defined user triggers are present by comparing the user input data to the stored set of defined user triggers.

At least some of the defined user triggers may have been defined automatically by circuitry of the apparatus 10 or of a further apparatus. At least some of the defined user triggers may have been defined by a user, who may be different from the user performing the one or more actions. The defined user triggers are individual action items that have been identified as being potentially relevant to determining one or more alerts. Further examples of defined user triggers and their use are provided below.

In the embodiment of FIG. 2, some user actions comprise defined user triggers, while other user actions do not comprise defined user triggers. For example, opening a checklist may be a defined user trigger, while closing the checklist may not be a defined user trigger.

If the one or more actions of stage 32 have been determined at stage 34 to comprise at least one defined user trigger, the method of FIG. 2 proceeds to stage 38, at which mapping of the defined user trigger onto a knowledge graph is performed as described below.

At stage 40, the trigger circuitry 26 processes at least part of the medical information contained within the EHR to determine whether the EHR comprises at least one defined trigger. Defined triggers obtained from the EHR may also be referred to as defined patient triggers, or as cues within the EHR data. In the present embodiment, a set of defined patient triggers are stored in the apparatus, for example, in the data store 20 or in any suitable data store. The trigger circuitry 26 determines whether any defined patient triggers are present by processing the EHR using the stored set of defined patient triggers.

At least some of the defined user triggers may have been defined automatically by circuitry of the apparatus 10 or of a further apparatus. At least some of the defined user triggers may have been defined by a user, who may be different from the user performing the one or more actions. The defined patient triggers are items of patient information that have been identified as being potentially relevant to determining one or more alerts.

In the present embodiment, patient triggers and user triggers are considered separately. In other embodiments, a single set of triggers may be defined which comprises both patient triggers and user triggers.

A defined patient trigger may be data representative of, for example, an event such as stroke or heart failure; an admission to hospital; a transfer between specialties, for example from general medicine to cardiology; or a consultation with a specified healthcare professional, for example orthopedics. A defined patient trigger may comprise one or more items of demographic information about the patient. A defined patient trigger may comprise a medication or treatment that has been prescribed to the patient, for example within a defined date range. A defined patient trigger may be found by parsing text in the patient's EHR. The presence of a defined patient trigger may be ascertained by searching unstructured and/or structured data in the patient's EHR. Further examples of defined patient triggers and their use are provided below.

If the EHR has been determined at stage 40 to comprise a defined patient trigger, the method of FIG. 2 proceeds to stage 38, at which an intelligent mapping of the defined patient trigger is performed as described below.

If no defined user trigger has been identified at stage 34 and no defined patient trigger has been identified at stage 40, the method of FIG. 2 proceeds to stage 42. At stage 42, a normal workflow is continued. For example, the clinical user may continue perform actions such as opening and reviewing documents, images or other data in the user interface.

If at least one defined user trigger has been identified at stage 34 and/or at least one defined patient trigger has been identified at stage 40, the method of FIG. 2 proceeds to stage 38. The at least one defined user trigger and/or at least one defined patient trigger may be referred to as trigger information.

At stage 38, the trigger circuitry 26 performs a mapping process in which each defined user trigger identified at stage 34 and each defined patient trigger identified at stage 40 is mapped onto a knowledge graph.

In the embodiment of FIG. 2, the knowledge graph is the UMLS (Unified Medical Language System) vocabulary. The knowledge graph comprises a plurality of nodes, each of which is representative of a respective concept having a corresponding concept unique identifier (CUI). The nodes are connected by edges. Each edge is representative of a relationship between the two nodes that it connects. Many different relationships may be represented by different edges of the knowledge graph. For example, a relationship between nodes may be is_a, inverse_isa, has_member, has_part, may_treat, cause_of, associated_symptom_of, or any suitable relationship.

The knowledge graph may be considered to be representative of a concept space in which related concepts are closer together (for example, connected by a lower number of edges) than unrelated concepts.

The knowledge graph may be navigated by identifying a concept and its children, where children of a concept are concepts that are contained within, but narrower than, the original concept. If the concept is a disease, its children may be disease subtypes. For example, for the concept of inflammatory bowel disease, children may include Crohn's disease and ulcerative colitis.

Each concept may be expressed by multiple different words or phrases, which may be described as surface forms. For example, ‘paracetamol’, ‘acetaminophen’ and ‘apap’ are different surface forms of the same concept, because they have equivalent meanings.

In other embodiments, any suitable knowledge graph may be used to store medical knowledge. In some embodiments, a different concept space may be used, which may not be UMLS. Any suitable knowledge base may be used to store information about concepts and their relationships, for example any suitable database, knowledge graph or ontology.

In the mapping process of stage 38, each defined user trigger identified at stage 34 and each defined patient trigger identified at stage 40 is mapped onto a knowledge graph by mapping the defined trigger to a corresponding concept. For example, a user action of reviewing a hip X-ray may provide a defined trigger of hip X-ray which is mapped to the clinical specialty of rheumatology. A patient trigger that is indicative of an event such as a stroke (e.g., the patient is referred to the stroke team) may be mapped to a concept that corresponds to that event.

In some circumstances, a user trigger comprising a search term may be mapped directly to a concept that corresponds to that search term. For example, a search term of ‘myocardial infarction’ may be mapped directly to the concept of myocardial infarction in UMLS.

In other circumstances, the exact search term may not be present in UMLS. The mapping of the search term onto the knowledge graph may be based at least in part on at least one of: fuzzy matching using an edit distance such as a Levenshtein edit distance; phonetic matching using a matching algorithm, for example Metaphone; stemming; and/or dictionary look-up of abbreviations. A machine learning model may be used to determine one or more related terms, for example a model as described in U.S. patent application Ser. No. 17/011,363, which is hereby incorporated by reference.

In the mapping process of stage 38, the trigger circuitry 26 also determines whether it is necessary to trigger any alert based on the defined user trigger(s) and/or defined patient trigger(s). The determining of whether it is necessary to trigger any alert may be described as determining a necessity of alerting. The determining of the necessity of alerting is based on the mapping of the trigger information onto the knowledge graph.

A set of rules is stored in the data store 20. Each rule of the set of rules describes the triggering of an alert in response to a sequence of one or more hits in concept space. A hit may refer to a mapping of a defined trigger onto a specified single CUI (node), or a mapping of a defined trigger into a pre-identified group of CUIs (nodes). The pre-identified group of CUIs defines a predetermined region of concept space, which may also be referred to as a subgraph or subregion of the knowledge graph. The predetermined region of concept space may comprise any suitable number of CUIs (nodes).

It is a feature of the method of FIG. 2 that the pre-defined group of CUIs may be defined using fuzzy logic. In one embodiment, a pre-defined group of CUIs is defined as cardiovascular CUIs. The group of cardiovascular CUIs may be described using fuzzy logic to include all terms relating to cardiovascular disease. In another embodiment, a pre-defined group of CUIs is defined to include medications used to treat asthma. In a further embodiments, a pre-defined group of CUIs is defined to include myocardial infarction and its synonyms.

The predetermined region of concept space may have fuzzy boundaries. Different parts of the predetermined region of concept space may have differing relevance.

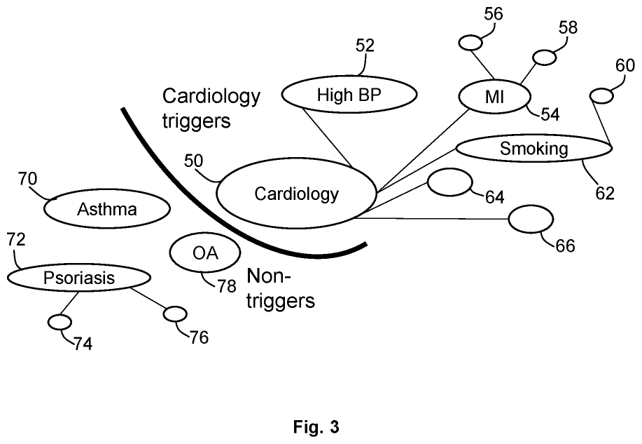

FIG. 3 is a schematic illustration showing a simplified example of nodes that form part of, and nodes that do not form part of, a pre-defined group of CUIs. A first set of nodes 50 to 66 may be considered to be cardiology triggers. For example, the first set of nodes comprises a node 50 that is representative of the concept of cardiology, a node 52 that is representative of the concept of high blood pressure (high BP), a node 54 that is representative of the concept of myocardial infarction (MI), and a node 62 that is representative of the concept of smoking. Other nodes 56, 58, 60, 64, 66 do not have their concepts explicitly shown in FIG. 3 but are also relevant to cardiology.

A second set of nodes 70 to 78 in FIG. 3 are not related to cardiology according to the definition of the pre-defined group of CUIs. For example, node 70 is representative of the concept of asthma. Node 72 is representative of the concept of psoriasis. Node 78 is representative of the concept of osteoarthritis (OA). Other nodes 74, 76 do not have their concepts explicitly shown in FIG. 3 but are also considered not to be relevant to cardiology.

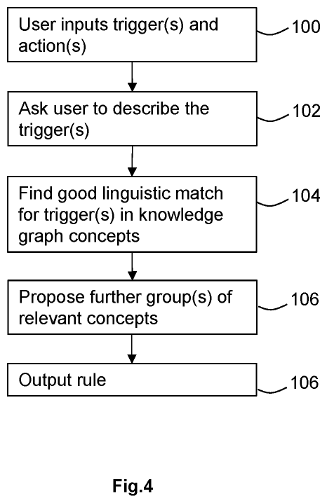

Each rule in the set of rules comprises a definition of a predetermined sequence that triggers the alert, and a description of the alert that is to be triggered by the predetermined sequence. Each defined trigger that forms part of the predetermined sequence may be described as an individual action (for example, viewing a cardiology letter) or as part of a class of actions (for example, any cardiology-related action). The sequence that triggers the alert may have been defined manually by a user, or may be defined automatically or semi-automatically, for example as described below with reference to FIG. 4.

Some rules of the set of rules may be considered to be simple rules relating triggers o alerts. Other rules of the set of rules may be considered to provide a smart mapping which may use, for example, a combination of simple rules with natural language processing and/or smart knowledge graph navigation.

In a first example of a rule (Rule 1), the predetermined sequence comprises a user searching for ‘myocardial infarction’ or a related term and the user viewing a letter classified as cardiology and/or viewing troponin lab results and/or viewing an ECHO (echocardiogram) image. The associated alert comprises retrieving and presenting cardiovascular risk factors from a patient's EHR or displaying local or national management guidelines.

A mapping logic for Rule 1 could be implemented as:
IF user searches for a term relating to ‘myocardial infarction’
AND does 1 or more of:
A) View letter classified as cardiology (e.g. using trained document classification model)
B) View troponin lab results
C) View ECHO image
THEN
Display cardiovascular risk factors detected in the patient's EHR using NLP
Display the local and national management guidelines for myocardial infarction

The step of searching for a term that is related to ‘myocardial infarction’ may comprise detecting a search term that is related to the concept of ‘myocardial infarction’ as defined in UMLS or is related to any of its descendant concepts according to inverse_isa or narrower concept relationships. The detecting of the search term may comprise using a semantic search function to find linguistic variants, for example synonyms, misspellings, abbreviations and shorthand.

In second example of a rule (Rule 2), the predetermined sequence comprises a patient admission in which a patient is admitted and referred to a stroke team. The alert comprises displaying a thrombolysis checklist panel.

In a third example of a Rule (Rule 3), the predetermined sequence comprises a user inputting a search term for ‘osteoarthritis’ and the user reviewing a hip X-ray image. The alert comprises highlighting pain medications or other rheumatology management in the EHR.

In a fourth example of a rule (Rule 4), the predetermined sequence comprises the user prescribing lisonipril, which is a blood pressure medication. The action comprises presenting a pop-up alert for any contraindications found in the EHR, for example a past medical history of asthma.

In some rules, the sequence is defined as the presence of one or more predefined actions in the absence of another predefined action. For example, the predetermined sequence may comprise opening a cardiology letter without checking a new blood result such as troponin or INR. If the user opens a cardiology letter without checking a new blood result, a corresponding alert may be triggered to display the new blood result to the user or to remind the user to check the new blood result. By detecting a specific non-action by the user in combination with other relevant actions by the user, the user may be alerted when something might have been missed.

In some rules, a relevance of alerting is determined based on a distance between a node corresponding to the alerting and a node corresponding to one or more triggers on the knowledge graph. If the shortest valid path between the two nodes is long (i.e., comprises many edges) this suggests that relevance is lower, and vice versa.

For each rule of the set of rules, the trigger circuitry 26 uses the mapping of the defined triggers onto the concept space to determine whether the predetermined sequence has been met. If the predetermined sequence has been met, the trigger circuitry 26 identifies that a corresponding alert is to be triggered.

If the trigger circuitry 26 has identified at stage 38 that an alert is to be triggered, the method of FIG. 2 proceeds to stage 44.

At stage 44, the trigger circuitry 26 passes information about the alert to the interface circuitry 24. The interface circuitry 24 executes the alert within the user interface, which may be referred to as a cockpit user interface.

The alert may comprise a user notification. For example, the user notification may comprise displaying an icon, and the user may interact with the icon, for example by clicking or mousing over the icon, to display relevant information. Information may be summarized in a pop up, tool tip or other display. The alert may be provided within an existing panel of the user interface. For example, the interface circuitry 24 may highlight relevant words in a panel of the user interface, such as ‘smoker’, ‘MI’ or ‘warfarin’. Alternatively or additionally, the alert may be provided within a new panel, for example when presenting a document to the user. Alternatively or additionally, the alert may be provided as a pop-up over an existing panel, for example highlighting an allergy.

In some embodiments, an alert comprises retrieving risk factors related to one or more defined triggers that have been used to trigger the alert, and presenting the risk factors to the user. For example, information regarding a risk factor of smoking status may be retrieved from the EHR and presented to the user as a highlighted term and/or as a pop-up.

In further embodiments, an alert comprises retrieving one or more documents relating to one or more defined triggers that have been used to trigger the alert and highlighting information within the documents, for example relevant words. For example, documents relating to pain medication may be retrieved and relevant words may be highlighted in the document. In the example of documents relating to pain medication, the words that are highlighted may comprise, for example, words such as codeine, morphine, WHO pain ladder.

In further embodiments, the alert comprises a prompt for a medication prescription or order.

In further embodiments, the alert may comprise displaying information in response to one or more defined triggers. For example, the interface circuitry 24 may display on the display screen 16 a link to local or national guidelines relating to the one or more user triggers. For example, NICE (National Institute for Health and Care Excellence) guidelines for hypertension management may be displayed.

Any of the above alerts may be displayed as a pop-up display, as a new panel, or in any suitable format.

In further embodiments, a pop-up or other notification may be used to highlight relevant factors, for example red flags or contraindications. For example, contraindications to tPA (tissue plasminogen activator) may be displayed. Information regarding a low eGFR (estimated glomerular filtration rate) or poor renal function may be displayed when a CT scan with contrast is requested.

In some embodiments, a pop-up or other notification may be used to highlight a required action that the user has not yet performed. A pop-up may be used to highlight what is potentially missed action by the user, for example checking new blood results such as troponin or INR (international normalized ratio) results.

Once any alert has been performed, the method of FIG. 2 proceeds to stage 46. At stage 42, a normal workflow is continued. For example, the clinical user may continue perform actions such as opening and reviewing documents, images or other data via the user interface.

The method of FIG. 2 may be repeated, for example when the user performs further actions.

The method of FIG. 2 may allow real-time configuration of the user interface to help the user in their specific clinical context, based on their actions within the cockpit. The method may allow faster, more efficient searching. The chances of missing clinically relevant information may be reduced. More comprehensive patient care may be provided.

Alerts may be provided that could, for example, collate risk factors, highlight red flags, provide a reminder of drug interactions and/or allow linking to clinical guidelines.

In some circumstances, it may be possible to identify information that doctors may not pick up on. Use of the EHR may be maximized while being time efficient.

The EHR is a rich data source that often contains the information required to infer that a clinical action is relevant. A method is proposed to prompt the doctor with relevant information or suggested actions in response to a relevant trigger, where the trigger may comprise an action or sequence of actions within the EHR. Triggers may be either patient- or user-related.

The use of rules may allow information to be shown proactively in response to various triggers. For example, in the case of Rule 1 as describes above, a search for the term ‘myocardial infarction’ and viewing of a cardiology letter are detected by the trigger circuitry 26 as multiple cardiology related actions, which is indicative of cardiology care. This may allow the trigger circuitry 26 to show relevant cardiovascular patient history as identified in UMLS. The interface circuitry 24 may proactively shown information required for a cardiology patient review. By displaying guidelines within the cockpit, all required information may be brought into the cockpit for ease of user.

In the case of Rule 2, the trigger circuitry 26 maps a stroke admission to a tPA checklist. The displaying of the tPA checklist may facilitate fast and safe administration of tPA where appropriate.

In the case of Rule 2, the trigger circuitry 26 detects that that ‘osteoarthritis’ & ‘hip x-ray’ are multiple rheumatology related actions, and determines that the presence of multiple rheumatology related actions is indicative of rheumatology care. Most osteoarthritis consultations are for pain review or surgical decision making, so in response to the multiple rheumatology actions, the trigger circuitry 26 may search for rheumatology management in the patient's EHR using concepts identified from UMLS. The trigger circuitry 26 retrieves details of pain medications or other rheumatology management in the patient's EHR. The interface circuitry 24 highlights the retrieved pain medications or other rheumatology management in the EHR, for example by showing a text document with highlighted words or phrases. Information required for rheumatology patient review is proactively shown to the user.

In the case of Rule 4, the trigger circuitry 26 detects that lisinopril is a medication for which UMLS links contra-indications that can be searched for in the patient's EHR. In response to the prescribing of lisinopril, the trigger circuitry 26 triggers a pop-up alert for contraindications. For example, the trigger circuitry 26 may search the patient's EHR and display alerts for asthma past medical history. By displaying an alert for asthma to the user, an inappropriate prescription may be avoided in some circumstances.

In each of Rules 1 to 4, the user is provided with additional information which may assist with the user's workflow.

Any suitable rules may form part of the set of rules that is stored and is used at stage 38. A rule may associate any suitable predetermined sequence of triggers with any suitable alert.

In the embodiment of FIG. 2, the trigger circuitry 26 identifies predefined triggers and then consults the set of rules to see whether any of the predefined triggers are relevant to the set of rules. In other embodiments, the stages of FIG. 2 may be performed in any order. For example, the trigger circuitry 26 may consult one or more rules first and then process the EHR and/or user actions to determine whether any of the triggers specified by the rules are present. The trigger circuitry 26 may obtain predefined user triggers from one or more user actions; consult a rule; and then process the EHR or other medical data to obtain predefined patient triggers.

The stages of FIG. 2 are performed using the apparatus 10. In other embodiments, one or more stages of FIG. 2 may be performed on one or more further apparatuses. In some embodiments, one or more stages of FIG. 2 are performed using cloud-based resources. In some embodiments, the knowledge graph or other knowledge base is stored in the cloud.

In some circumstances, a rule may comprise one or more parameters and/or may comprise a quantification step. For example, a predetermined sequence may comprise a number of user actions required to trigger an alert. A rule may define a relative importance of different triggers. For example, the rule may comprise a ranking of importance of different constituent triggers for a given mapping to an action. In some embodiments, a user is asked to provide a relevance score or ranking for each trigger.

In some circumstances, knowledge graphs and/or NLP techniques are used not only to identify relevant triggers but to quantify their relevance. Relevance may be quantified using a distance from an original concept in a knowledge graph. Relevance may be quantified using a classification probability in a text classification model, which may provide a probability of a match. In other embodiments, relevance may be determine using any suitable method.

In some embodiments, one or more parameters associated with a rule are used to provide the user with control over the application of that rule. The parameters may comprise for example, one or more measures of relevance (for example, a distance and/or a classification probability), one or more measures of importance and/or a number of user actions. The trigger circuitry 26 combines values for the parameters to obtain a ratio of false positives to false negatives for the rule given different values for the parameters. A combination of parameters may be performed using simple arithmetic to combine aspects such as the number of triggers, or a number of different triggers. A relevance score or ranking provided by a user may inform a weighted calculation for different alert thresholds.

In an embodiment, the trigger circuitry 26 allows the user to select a ratio of false positives to false negatives. When the user selects the ratio, the trigger circuitry 26 adjusts the values for the parameters to obtain the user's selected ratio. The user may therefore control the sensitivity and specificity of the alerts. In other embodiments, any suitable method may be used to control the sensitivity and/or specificity.

An operating point may be controlled using a number of triggers or a number of different triggers. An operating point may be controlled by a probability of trigger detection, in cases in which detection of triggers is performed with machine learning or other methods with a non-binary detection output.

A ratio of true positive rate to false positive rate may be described as a ROC (receiver operating characteristic) curve. By allowing the user to adjust a ratio of false positives to false negatives, the trigger circuitry 26 allows the user to adjust an operating point on the ROC curve. In some circumstances, the operating point may be adjusted to decrease sensitivity to prevent alert fatigue. In some circumstances, the operating point may be adjusted to increase sensitivity to provide more support to a user.

Any suitable input method may be used to allow the user to perform adjustments. For example, a slider may be presented in the user interface which allows the user to select higher or lower sensitivity. In other embodiments, any suitable selection component may be presented to the user to allow the user to select higher or lower sensitivity and/or specificity. For example, the selection component may comprise a slider, a text box, a drop-down menu, or one or more buttons or checkboxes.

In some embodiments, the user can adjust all of the rules. In other embodiments, the user can adjust only some of the rules, for example those rules that include a relevance or a number of actions.

In the embodiment of FIG. 1, the apparatus 10 is configured to create rules that map triggers to actions as well as to execute such rules in response to inputs. In other embodiments, the apparatus that creates the rules is different from the apparatus 10 that executes the rules. Rules may be created automatically, semi-automatically or manually. A process of rule creation is now described with reference to FIG. 4.

FIG. 4 is a flow chart illustrating in overview a method of rule creation in accordance with an embodiment. The rule creation is performed using inputs from a clinician or from another workflow designer. The training circuitry 28 provides prompts to the clinician or other workflow designer to assist the rule creation.

At stage 100, the clinician or other workflow designer inputs a description of one or more relevant triggers and a corresponding action to be performed in response to the relevant trigger(s). The corresponding action may also be referred to as an alert.

In one example, the trigger provided by the user is ‘myocardial infarction’, and the action provided by the user is to display clinical guidelines for managing myocardial infarction. The interface circuitry 24 passes data representative of the user's input to the training circuitry 28.

At stage 102, the training circuitry 28 instructs the interface circuitry 24 to provide a display asking the user to further describe the trigger(s). In one example, the interface circuitry 24 displays the following set of questions:
Trigger is user search query
   How many times?
Trigger is mention in text of patient data
   Date
      Just arrived?
      Since admission?
      Within last . . . months?
   Which data?
      Patient notes?
      Image metadata?

The user inputs answers to the questions via the user interface. For example, if the trigger comprises a user search query, the display asks the user how many times a user search query within an appropriate scope must occur for the trigger to be met. If the trigger comprises a mention in the text of the patient data included in the EHR, the display asks the user for date parameters (just arrived, since admission and/or within a number of months) and what parts of the EHR should be searched (patient notes and/or image metadata). In other embodiments, any other suitable question or request for data may be displayed to the user for the user to provide user input.

The user input received at stage 102 may allow the training circuitry 28 to define the trigger in more detail than is provided in the user's original request at stage 100. By providing prompts to the user, the user may be guided towards a suitable trigger definition.

At stage 104, the training circuitry 28 finds a good linguistic match for the trigger in the knowledge graph concepts. The training circuitry 28 maps the user's description of the one or more relevant triggers onto a knowledge graph. In some embodiments, the user may specify a concept, for example a UMLS concept. In other embodiments, the training circuitry 28 automatically identifies one or more concepts, for example by matching a key term specified by the user to identify a closest matching term and concept in the knowledge graph using edit distance (fuzzy string matching). In some embodiments, one or more concepts may be obtained using at least one of: fuzzy matching using an edit distance such as a Levenshtein edit distance; phonetic matching using a matching algorithm, for example Metaphone; stemming; and/or dictionary look-up of abbreviations.

In the embodiment of FIG. 4, the training circuitry 28 finds in the UMLS knowledge graph a set of best matches for the trigger term that was input by the user. The training circuitry 28 then instructs the interface circuitry 24 to request the user to rate the best matches. The user inputs relevance scores which the interface circuitry 24 passes to the training circuitry.

For example, for the trigger ‘myocardial infarction’, the training circuitry finds three UMLS concepts that could be relevant to myocardial infarction. The first concept is Myocardial infarction (C0027051)—disease. The second concept is Electrocardiogram: myocardial infarction (C0428953)—laboratory or test result. The third concept is Family history: myocardial infarction (C0455406)—finding etc. The training circuitry 28 instructs the interface circuitry 24 to display to the user details of the three concepts and to ask the user to assign a respective relevance score to any of the first, second and third concepts that apply to the trigger selected by the user. In one example, the user assigns a relevance score of 5 to the first concept, assigns a relevance score of 3 to the second concept, and does not assign any relevance score to the third concept.

At stage 106, the training circuitry 28 uses the relevance scores provided by the user to propose further groups of relevant concepts, which are related to the concepts that the user indicated were relevant in stage 104. Further groups of relevant concepts may be identified by traversing the knowledge graph according to predefined traversal rules specified by the user or by the mapping method. A traversal of the knowledge graph may start at a concept, which may be referred to a starting concept. The traversal may be informed by features of the starting concept.

Traversal rules may be dependent on a semantic type of the starting concept, such that different traversal rules are applied to starting concepts of different semantic types. For example, different traversal rules may be applied to a medication than to a disease.

Traversal rules may be dependent on a node level of the starting concept, such that different traversal rules are applied to starting concepts having different node levels. For example, the hypernyms (i.e., parent concepts) of a high-level node are usually not closely related terms. Therefore in some embodiments navigation through the knowledge graph would omit upward is_a relationships if the starting concept is a high-level node.

Traversal rules may be informed by a sequence of relationship traversals. For example, for a medication, if the has_active_ingredient edge is followed, then a subsequent traversal may omit active_ingredient_of edges. For instance, the medication co-codamol contains both paracetamol and codeine. If co-codamol is searched for, then paracetamol is a relevant finding. However, Lemsip (for which paracetamol is also an active ingredient) would not be a relevant finding in relation to co-codamol.

Traversal rules may be informed by distance in the graph, for example a number of relationships (edges). For example, traversal may cease after a predetermined number of edges.

Traversal rules from a given node may be dependent on a connectivity of that node, where a number of edges connected to a node denotes its connectivity.

In other embodiments, any suitable traversal rules may be used to traverse the knowledge graph or other knowledge base. A machine learning model may be used to determine one or more related terms, for example a model as described in U.S. patent application Ser. No. 17/011,363, which is hereby incorporated by reference.

The training circuitry 28 instructs the interface circuitry 24 to display the proposed relevant concepts to the user so that the user can select any of the proposed relevant concepts that they consider to be relevant to the trigger. In other embodiments, any suitable number of proposed relevant concepts may be displayed in any suitable manner, which may not comprise displaying as further groups.

In the example in which the trigger is ‘myocardial infarction’, the interface circuitry 24 asks the user:
Would you like all descendants of myocardial infarction? Can you assign relevance scores if different to parent concept?

The interface circuitry 24 displays a set of concepts that are descendants of myocardial infarction in a UMLS knowledge graph. For example, Acute MI and Postoperative MI are both descendants of the concept Myocardial infarction C0027051. The user may select all descendants of Myocardial infarction C0027051. If the user considers that any of the descendants of Myocardial infarction C0027051 have higher or lower relevance than Myocardial infarction C0027051, the user may provide a relevance score for such descendants.

The training circuitry 28 may use graphs and/or other natural language processing (NLP) techniques to identify groups of relevant terms. The training circuitry 28 may perform a quantification of a degree of relevance. For example, if an initial term is identified by a user, the training circuitry 28 may determine a relevance of a related term based on a distance of the related term from the initial term, for example as a number of edges in the knowledge graph. The training circuitry 28 may determine a relevance of the related term based on a type of relationship between the initial term and the related term.

By using graphs and other NLP techniques to identify groups of relevant terms, a set of hits may be defined in a way which may be described as fuzzy. Fuzzy logic may be used to implement triggers such as ‘all terms relating to cardiovascular disease’, ‘myocardial infarction and its synonyms’ or ‘medications for asthma’. A subregion of the knowledge graph may be defined, which may be referred to as a subgraph. The subgraph may have fuzzy boundaries. Relevance may decrease when approaching an edge of the subgraph.

By obtaining user input at stage 104 and/or stage 106, a rule may be better defined. The training circuitry 28 may also ask questions to further define the action requested by the user (not shown in FIG. 4).

At stage 108, the training circuitry 28 outputs a rule that comprises a predetermined sequence of defined triggers, and at least one corresponding alert. The rule may be stored as part of a set of rules. The rule may be applied using the method of FIG. 2.

In other embodiments, the training circuitry 28 is configured to automatically infer one or more rules by detecting common sequences of triggers and user actions that are performed by one or more users. If classes of different actions have already been defined, the training circuitry 28 may identify different triggers to be included in those classes.

The training circuitry 28 may recognize that different actions may be considered as a common trigger. For example, search terms, terms in patient text and reports may all map to the same part of UMLS space.

The training circuitry 28 may detect common patterns and propose one or more new rules to a clinician based on the detected patterns. A pattern may comprise a series of actions performed by a user. A pattern may comprise the absence of an action, for example a user not giving tPA in response to a stroke.

The training circuitry 28 may augment or refine one or more existing trigger to alert mappings by tracking additional similar cases where a user takes an action. The training circuitry 28 may augment or refine one or more existing trigger to alert mappings by tracking existing cases where a user chooses not to take an action.

For example, the training circuitry 28 may detect that a user never uses an alert that is displayed. The training circuitry 28 may suggest a modification to a rule so that the alert is no longer displayed.

The training circuitry 28 may generate rules automatically or semi-automatically by using any suitable learning process to perform an automatic inference.

In some embodiments, the training circuitry 28 may make use of a bank of possible follow-on actions from which alerts can be created. The training circuitry 28 may use information about possible follow-on actions to infer or adjust mapping rules. The training circuitry 28 may detect common sequences of trigger actions and follow-on actions. The training circuitry 28 may detect a common sequence of similar trigger actions and follow-on actions. Similar trigger actions and follow-on actions may be detected using fuzzy concept matching and linking to recognize where triggers are similar, for example by traversing a knowledge graph in accordance with traversal rules. The training circuitry 28 may detect cases in which there is a similar trigger action to follow-on action sequence to an existing mapping rule, which the existing mapping rule does not contain. The training circuitry 28 may detect cases where existing mappings do not lead to a user response, for example cases in which an alert is ignored.

The training circuitry 28 may instruct the interface circuitry 24 to present proposed rules to a user for the user's approval or adjustment.

In the above embodiments, information is obtained from an EHR relating to a patient. In other embodiments, a corresponding method may be performed on any suitable medical data for any human or animal subject.

Certain embodiments provide a medical information processing system comprising: a memory stores a knowledge graph based on medical knowledge, and: processing circuitry configured to: receive a trigger information, determine a necessity of alerting relating to the trigger information based on the trigger information and the knowledge graph.

The processing circuitry may be further configured to determine the necessity of alerting by mapping the trigger information on the knowledge graph.

The processing circuitry may be further configured to: decide the necessity of the alerting based on the relevance.

The trigger information may be determined based on patient information of target patient.

The trigger information may be determined based on an action of user relating to a target patient.

The knowledge graph may be ontology based on a target patient.

Certain embodiments provide a semi-automated process that works within a clinical decision support system. Semi-automated creation of a trigger->alert mapping between medical concepts which enables us to: detect cues in the patient's EHR data, or user actions; trigger an intelligent mapping process; and then alert the user to a possible action; whilst an authorised clinical user interacts with one patient's EHR.

Certain embodiments provide a method to trigger alerts within a clinical decision support system comprising: detection of patient data cues or user actions; a medical knowledge graph/ontology; an expanded subgraph in the ontology, mapping the extent of the relevance domain; a user or workflow defined action; in which the data cues and user actions are mapped into the expanded relevance domain and tracked until the trigger condition is fulfilled whereby the trigger action is performed.

The triggers may comprise at least one of: parsing text in the patient's EHR; detecting sequences of search inputs; detecting document opening; detecting image review.

The alerts may comprise at least one of: retrieval and presentation of information from the EHR (e.g. retrieving a relevant document); retrieval and highlighting of information within the EHR (e.g. highlighting words within a relevant document(s)); display of information (e.g. clinical guidelines); pop-up that highlights important information (e.g. an allergy); prompt for medication prescription/order.

The mapping process may use concepts in a medical terminology or knowledge graph, the concepts comprising at least one of: user-selected concepts; automatically identified concepts, by matching the users specified key term to the closest matching term (and concept) in the graph e.g. using edit distance (fuzzy string mapping); further relevant groups of concepts identified by traversing the knowledge graph, according to predefined rules specified by the user or by the mapping method. Traversal rules may be informed by the semantic type of the starting concept. Traversal rules may be informed by the node level of the starting concept. Traversal rules may be informed by the sequence of relationship traversals. Traversal rules may be informed by distance in the graph e.g. number of relationships (edges). Traversal rules may be informed by connectivity of a node i.e. the number of edges connected to a node denotes its connectivity.

The mapping process may detect relevant concepts or keywords in medical text using natural language processing techniques, including machine learning techniques.

The mapping may allow the user to control the sensitivity and specificity of the alerts. The operating point may be controlled by the probability of trigger detection, in cases where components are detected with machine learning components or other methods with a non-binary detection output. Mapping components may be combined using simple arithmetic of aspects of the mapping such as the number of triggers, or number of different triggers. The user might be asked to provide a relevance score or ranking for each trigger, which can then inform a weighted calculation for different alert thresholds.

The mapping may be inferred or adjusted by the system, using a bank of possible follow-on actions for which alerts can be created: by detecting common sequences of trigger actions and follow-on actions; by detecting common sequence of similar trigger actions and follow-on actions, using fuzzy concept matching and linking, to recognise where triggers are similar; by detecting cases where there is a similar trigger action to follow-on action sequence to an existing mapping, which the mapping does not contain; and/or by detecting cases where existing mappings do not lead to a user response i.e. the alert is ignored.

Certain embodiments provide a method for triggering alerts within a clinical decision support system, the method comprising: receiving a trigger relating to an electronic health record for a patient, the trigger comprising at least one patient data cue and/or at least one user action; performing a mapping procedure to determine if the trigger is mapped to an alert, the mapping procedure comprising: accessing a medical knowledge graph or ontology; and determining, using the medical knowledge graph or ontology, whether the trigger falls within a domain of relevance for the alert; and if the trigger is mapped to an alert, presenting the alert to the user.

The at least one patient data cue may be obtained by analysing the electronic health record. The at least one patient data cue may be obtained by parsing text of the electronic health record.

The at least one user action may comprise at least one of a search input; a sequence of search inputs; opening of a document in the electronic health record; review of an image in the electronic health record; prescribing of a medication; filling out a checklist.

The domain of relevance may be defined with reference to a subgraph of the knowledge graph or ontology.

Presenting the alert to the user may comprise at least one of a) to e):—
a) retrieving and presenting information in the electronic health record (e.g. retrieving a relevant document);
b) retrieving and highlighting information in the electronic health record (e.g. highlighting words within a relevant document(s);
c) displaying information (e.g. clinical guidelines);
d) presenting a pop-up that highlights important information in the electronic health record (e.g. an allergy);
e) displaying a prompt for a medication prescription or order.

The method may further comprise defining the domain of relevance of the alert using concepts in the knowledge graph or ontology.

The method may further comprise obtaining a starting concept from a user selection. The method may further comprise obtaining a starting concept automatically by matching a key term specified by a user to a closest matching term and/or closest matching concept in the knowledge graph or ontology. The matching may be performed using an edit distance. The matching may be performed using fuzzy string matching.

The defining of the domain of relevance may comprise identifying relevant groups of concepts by traversing the knowledge graph or ontology according to predefined rules specified by the user and/or by the mapping method.

Traversal rules may be informed by a semantic type of the starting concept. Traversal rules may be informed by a node level of the starting concept. Traversal rules may be informed by a sequence of relationship traversals. Traversal rules may be informed by distance in the graph e.g. number of relationships (edges). Traversal rules may be informed by connectivity of a node, wherein the number of edges connected to a node denotes its connectivity.

The method may further comprise identifying relevant concepts or keywords in medical text using natural language processing techniques, including machine learning techniques.

The method may further comprise changing the domain of relevance in response to a user input, thereby controlling a sensitivity and specificity of the alert.

The method may further comprise controlling an operating point by a probability of trigger detection, for example, in cases where components are detected with machine learning components or other methods with a non-binary detection output.

The user input may comprise selecting a number of triggers or a number of different triggers. The user input may comprise a relevance score or ranking for each trigger. The relevance score or ranking may inform a weighted calculation for different alert thresholds.

The method may further comprise creating a new combination of trigger and alert by at least one of a) to d):
a) detecting common sequences of trigger actions and follow-on actions;
b) detecting common sequences of similar trigger actions and follow-on actions, using fuzzy concept matching and linking to recognise where triggers are similar.
c) detecting cases where there is a similar trigger action to follow-on action sequence to an existing mapping, which the mapping does not contain;
d) detecting cases where existing mappings do not lead to a user response i.e. an alert is ignored.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical information processing system for processing medical data relating to a target patient, the medical information processing system comprising:
a memory storing a knowledge base, wherein the knowledge base is based on medical knowledge; and
processing circuitry configured to:
receive trigger information; and
determine a necessity of alerting relating to the trigger information based on the trigger information and the knowledge base.

2. A medical information processing system according to claim 1, wherein the processing circuitry is further configured to issue an alert based on the determined necessity of alerting.

3. The medical information processing system according to claim 1, wherein the determining of the necessity of alerting comprises mapping the trigger information onto the knowledge base.

4. The medical information processing system according to claim 3, wherein the mapping of the trigger information onto the knowledge base comprises determining whether the trigger information falls within a predetermined subregion of the knowledge base.

5. The medical information processing system according to claim 1, wherein the knowledge base comprises at least one of a database, a knowledge graph, an ontology.

6. The medical information processing system according to claim 1, wherein the knowledge base comprises a knowledge graph and the processing circuitry is configured to determine a relevance of the trigger information based on a distance between at least one node of the knowledge graph that corresponds to the trigger information and at least one further node of the knowledge graph that corresponds to the alerting.

7. The medical information processing system according to claim 6, wherein the determining of the necessity of the alerting is based on the determined relevance.

8. The medical information processing system according to claim 1, wherein the processing circuitry is further configured to determine the trigger information based on patient information relating to the target patient.

9. The medical information processing system according to claim 8, wherein the patient information comprises an Electronic Health Record for the target patient.

10. The medical information processing system according to claim 1, wherein the processing circuitry is further configured to determine the trigger information based on an action of a user relating to the target patient.

11. The medical information processing system according to claim 10, wherein the action of the user comprises at least one of: one or more search inputs; a document opening; a document input; a checklist opening; a checklist input; an image review; a prescribing.

12. The medical information processing system according to claim 1, wherein the alerting comprises retrieving information in the medical data and presenting or highlighting the retrieved information.

13. The medical information processing system according to claim 1, wherein the alerting comprises at least one of a display, a highlighting, a pop-up, a prompt.

14. The medical information processing system according to claim 1, wherein the processing circuitry is further configured to present to a user a selection component for selecting a sensitivity and/or specificity of alerting, and wherein the determining of a necessity of alerting is in dependence on a sensitivity and/or specificity selected by the user.

15. The medical information processing system according to claim 14, wherein the processing circuitry is further configured to relate one or more parameters to sensitivity and/or specificity and to adjust the one or more parameters in dependence on the sensitivity and/or specificity selected by the user, the one or more parameters comprising at least one of: a number of triggers, a number of different triggers, a probability of classification.

16. A medical information displaying apparatus comprising the medical information processing system of claim 1 and a display screen, wherein:
the processing circuitry is further configured to display on the display screen an interface for accessing the medical data relating to the target patient;
the processing circuitry is further configured to receive at least one user input;
the receiving of the trigger information comprises processing the at least one user input and/or processing the medical data relating to the target patient; and
the alerting comprises displaying an alert on the display screen.

17. A method for processing medical data relating to a target patient, the method comprising:
receiving trigger information; and
determining a necessity of alerting relating to the trigger information based on the trigger information and a knowledge base, wherein the knowledge base is based on medical knowledge.

18. A medical information processing system for determining rules associating alerting with trigger information, the medical information processing system comprising:
a memory storing a knowledge base, wherein the knowledge base is based on medical knowledge; and
processing circuitry configured to:
receive or determine one or more concepts;
determine a subregion of the knowledge base based on the one or more concepts; and
associate one or more triggers within the subregion with an alert.

19. A medical information processing system according to claim 18, wherein the processing circuitry is further configured to detect a combination of trigger information with at least one following action and to determine the concept, the subregion and the alert based on the combination of the trigger information with the at least one following action.

20. A medical information processing method for determining rules associating alerting with trigger information, the method comprising:
receiving or determining one or more concepts;
determining a subregion of a knowledge base based on the one or more concepts, wherein the knowledge base is based on medical knowledge; and
associating one or more triggers within the subregion with an alert.
